(19) United States
(12) Patent Application Publication
(10) Pub. No.: US 2015/0290412 A1
(43) Pub. Date: Oct. 15, 2015

(54) Airway Opening Device

(71) Applicant: Shahriar Heydari, Rapid City, SD (US)

(72) Inventor: Shahriar Heydari, Rapid City, SD (US)

(21) Appl. No.: 14/672,767

(22) Filed: Mar. 30, 2015

Related U.S. Application Data
(60) Provisional application No. 61/977,858, filed on Apr. 10, 2014.

(51) Int. Cl. A61M 16/04; A61M 16/20

(52) U.S. Cl. CPC A61M 16/0434; A61M 16/0495; A61M 16/0497; A61M 16/201; A61M 2205/07; A61M 2205/02

(57) ABSTRACT

An airway opening device for maintaining an airway in a supine patient comprises a hard palate having a handle and a curved portion. The curved portion covers and supports the tongue of the supine patient. An inflating tubular component attached to the curved portion of the hard palate. The inflating tubular component further comprises an inflation tube and an inflation pump. The inflation pump attached to the inflation tube by means of tubing. The inflation pump having a pressure screw control valve prevents the air from escaping. The inflating tubular component pushes the curved portion of the hard palate against the tongue, thereby creating an airway.

### CROSS-REFERENCE TO RELATED APPLICATIONS

Not Applicable.

### STATEMENT REGARDING FEDERALLY SPONSORED RESEARCH AND DEVELOPMENT

Not Applicable.

### FIELD OF THE DISCLOSURE

This embodiment relates to medical devices and more particularly to an airway opening device in resisting collapse of the base of the tongue, thereby providing a clear airway passage to a patient and permitting to continue breathing while in unconsciousness.

### DISCUSSION OF RELATED ART

In an individual, an air passage is secured in the larynx even when a person sleeps on the back so that air can flow through the rear of the root of the tongue to permit breathing without difficulty. In a person with consciousness disorder, however, muscular tonus will disappear so that while sleeping in the supine position the root of the tongue will subside and thereby obstruct the passage of air, that is, the laryngeal airway with the result of causing breathing difficulty. This will inevitably result in a deterioration of the pathological condition. In such a case, it is desirable to have any device that is capable to provide air passage for facilitating an undisturbed breathing.

One such device provides a passage for air or anesthetic gas into the trachea. The airway is adapted to hold the mouth open to prevent excessive force being exerted between the upper and lower front teeth and to prevent damage to the teeth of the patient which can occur owing to involuntary clamping of the jaw during anesthesia. The described airway has bracing portions in the form of two-section tubes that is adapted to fit between the upper and lower molar teeth on each side of the mouth. The tubes also provide part of the passage for air or gas. The bracing tubes are connected to a third tube which is curved so as to extend into the orapharyngeal space. However, this device is small in size and causes irritation and pain in patients, particularly those with dental trauma injuries.

Another existing airway-clearing device discloses upper and lower elongated, substantially planar surfaces connected in a spaced relation by two rows of longitudinally-spaced apart struts on opposite sides of a central channel. Convergent inner surfaces of the struts guide a suction tube through the airway and the spaces between the struts allow for drainage of the central channel to prevent occlusion. However, such a device includes a relatively large number of parts, is expensive and difficult to operate.

In light of the foregoing, there is a need for an improved airway opening device to provide an unrestricted airway passage at the time a patient suffers from a consciousness disorder. Such an airway opening device would be a hybrid combination of laryngoscope and an oropharyngeal airway. Such an airway opening device would comprise a hard palate and an inflating tube. The hard palate would cover and support the tongue in place and the inflating tube would expand the hard palate and provide an effective air passage. The present embodiment accomplishes these objectives.

### SUMMARY OF THE DISCLOSURE

An airway opening device for maintaining an airway in a supine patient is disclosed. The airway opening device comprises a hard palate and an inflating tubular component. The hard palate comprises a handle and a curved portion. The inflating tubular component is attached to the curved portion of the hard palate. The inflating tubular component further comprises an inflation tube and an inflation pump attached to the inflation tube by means of tubing. The inflating tubular component pushes the curved portion of the hard palate against the tongue, thereby creating an airway.

The device is placed inside the mouth of an individual. Air is pumped into the inflation tube to tighten the inflation tube inside the mouth of an individual. Air is pumped into the inflation tube through the tubing and pressure screw control valve by squeezing or compressing the inflation pump. The thumb screw on the pressure screw control valve also must be closed for the air pumped by the inflation pump to inflate the inflation tube. If the thumb screw is not closed, then air will exit the pressure screw control valve through the thumb screw, rather than pass through the tubing to inflate the inflation tube. Opening the thumb screw will also release any air contained in the inflation tube or the tubing. With the thumb screw closed, air pumped by the inflation pump through the pressure screw control valve and the tubing causes the inflation tube to expand and tighten inside the patient's mouth. Air is continually pumped from the inflation pump into the inflation tube until a pressure level is reached. The curved portion in the hard palate covers and supports the tongue of the supine patient. There is a perpendicular crest at the junction of the curved portion and the handle. The perpendicular crest will lodge behind the front teeth (in front of the tongue) for better stabilization of the device when in operation. The inflating tubular component pushes the curved portion of the hard palate against the tongue, thereby creating an airway and a passage for visualization of oropharynx and ideally the vocal cords.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

FIG. 1 is a perspective view of an airway opening device in accordance with a preferred embodiment of the present invention;

FIG. 2 is a perspective view of a hard palate of the airway opening device in accordance with a preferred embodiment of the present invention;

FIG. 3 is a perspective view of an inflating tubular component of the airway opening device in accordance with a preferred embodiment of the present invention; and

FIGS. 4-5 illustrate the airway opening device in use.

### DETAILED DESCRIPTION OF THE PREFERRED EMBODIMENT

The following describes example embodiments in which the present invention may be practiced. This invention, however, may be embodied in many different ways, and the description provided herein should not be construed as limiting in any way. Among other things, the following invention may be embodied as methods or devices. As such, the present invention may take the form of an entirely hardware embodiment, an entirely software embodiment, or an embodiment combining software and hardware aspects. The following detailed descriptions should not be taken in a limiting sense.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

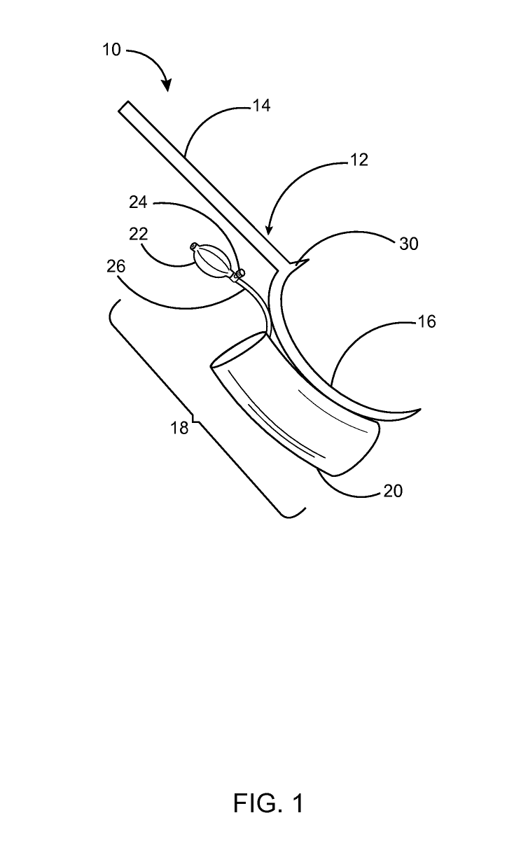

Referring to FIG. 1, a perspective view of an airway opening device 10 in accordance with a preferred embodiment of the present invention is illustrated. The airway opening device 10 comprises a hard palate 12 and an inflating tubular component 18. The hard palate 12 comprises a handle 14 and a curved portion 16. The inflating tubular component 18 is attached to the curved portion 16 of the hard palate 12. The inflating tubular component 18 further comprises an inflation tube 20 and an inflation pump 22 attached to the inflation tube 20 by means of a tubing 26. The inflating tubular component 18 pushes the curved portion 16 of the hard palate 12 against the tongue 32, thereby creating an airway.

The handle 14 in the hard palate 12 is a spoon shaped structure. The curved portion 16 in the hard palate 12 covers and supports the tongue 32 of the supine patient 28. A perpendicular crest 30 is provided at the junction of the curved portion 16 and the handle 14. The perpendicular crest 30 will lodge behind the front teeth (in front of the tongue 32) for better stabilization of the device 10 when in operation. The inflating tubular component 18 pushes the curved portion 16 of the hard palate 12 against the tongue 32, thereby creating an airway.

The inflation pump 22 plays a major role in creating an inner hollow cavity. The inflation pump 22 is preferably formed from a non-latex, polymeric material, such as polyvinyl chloride with a plasticizer for achieving a quick response from compressing and releasing the inflation pump 22. The inflation pump 22 is typically an oval bulb made of black rubber. The inflation pump 22 inflates the inflation tube 20 to stop the air flow for a few seconds. The inflation pump 22 injects air into the inflation tube 20.

The inflation pump 22 contains a pressure screw control valve (not shown) that prevents the air from escaping. The pressure screw control valve (not shown) is adapted to open and release pressure from the internal volume when the pressure is higher than a predefined pressure limit. Thus, the device 10 is adapted to detect when the internal volume is pressurized excessively and to spontaneously alleviate the impact of excess pressure if and when it is detected, thereby allowing the device 10 to be used with a high level of safety in the patient 28.

The device 10 for maintaining an oropharyngeal airway is effective in resisting the collapse of the base of the tongue 32. As with apnea and hypopnea during sleep, anesthesia or sedation can be caused by the collapse of the base of the tongue 32 into the oropharynx causing obstruction of the airway, the device 10 is therefore effective in maintaining the oropharyngeal airway at least sufficiently open in use so that the patient 28 is able to continue breathing. Preferably, the external pressure is applied by the oropharyngeal wall. Advantageously, the device 10 for maintaining an oropharyngeal airway is effective in resisting the collapse of the oropharyngeal wall and relaxation of the tongue 32 muscles.

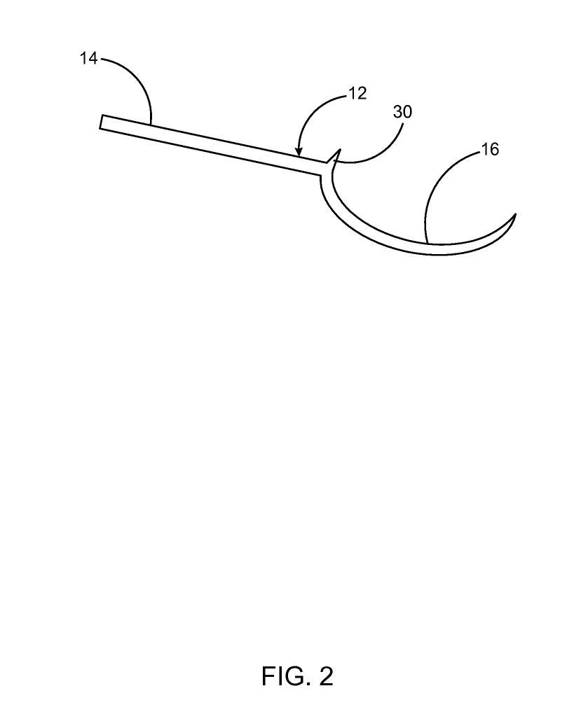

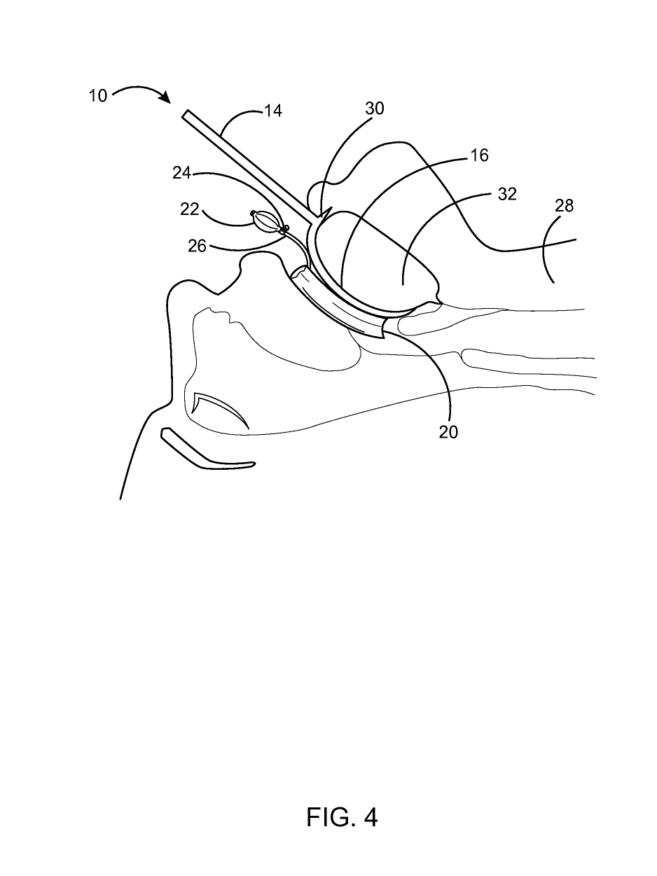

FIG. 2 is a perspective view of the hard palate 12 of the airway opening device in accordance with the preferred embodiment of the present invention. The airway opening device 10 comprises a hard palate 12 and an inflating tubular component 18. The hard palate 12 comprises a handle 14 and a curved portion 16. The handle 14 in the hard palate 12 is a spoon shaped structure. The curved portion 16 in the hard palate 12 covers and supports the tongue 32 (See FIG. 4) of the supine patient 28 (See FIG. 4). A perpendicular crest 30 is provided at the junction of the curved portion 16 and the handle 14. The perpendicular crest 30 will lodge behind the front teeth (in front of the tongue 32) (See FIG. 4) for better stabilization of the device 10 when in operation.

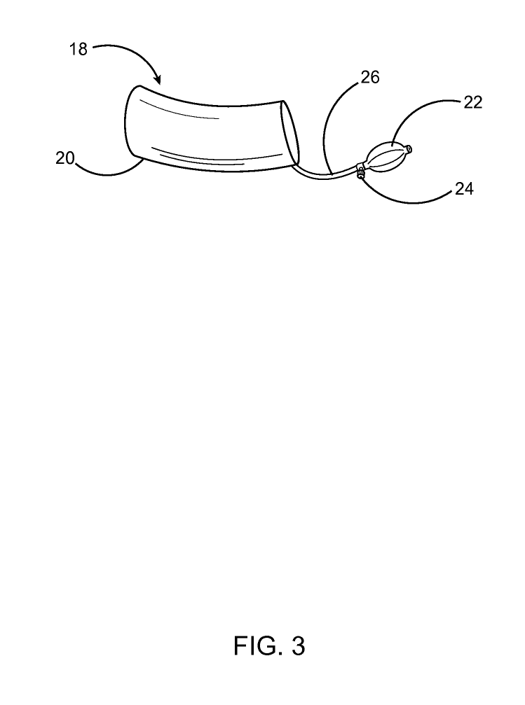

FIG. 3 is a perspective view of an inflating tubular component 18 of the airway opening device in accordance with the preferred embodiment of the present invention. The airway opening device 10 comprises a hard palate 12 and an inflating tubular component 18. The inflating tubular component 18 is attached to the curved portion 16 of the hard palate 12 The inflating tubular component 18 further comprises an inflation tube 20 and an inflation pump 22 attached to the inflation tube 20 by means of a tubing 26. The inflating tubular component 18 pushes the curved portion 16 of the hard palate 12 against the tongue 32 thereby creating an airway.

The inflation pump 22 plays a major role in creating an inner hollow cavity. The inflation pump 22 is preferably formed from a non-latex, polymeric material, such as polyvinyl chloride with a plasticizer for achieving a quick response from compressing and releasing the inflation pump 22. The inflation pump 22 is typically an oval bulb made of black rubber. The inflation pump 22 inflates the inflation tube 20 to stop the air flow for a few seconds. The inflation pump 22 injects air into the inflation tube 20.

The inflation pump 22 contains a pressure screw control valve (not shown) that prevents the air from escaping. The pressure screw control valve (not shown) is adapted to open and release pressure from the internal volume when the pressure is higher than a predefined pressure limit. Thus, the device 10 is adapted to detect when the internal volume is pressurized excessively and to spontaneously alleviate the impact of excess pressure if and when it is detected, thereby allowing the device 10 to be used with a high level of safety in the patient 28.

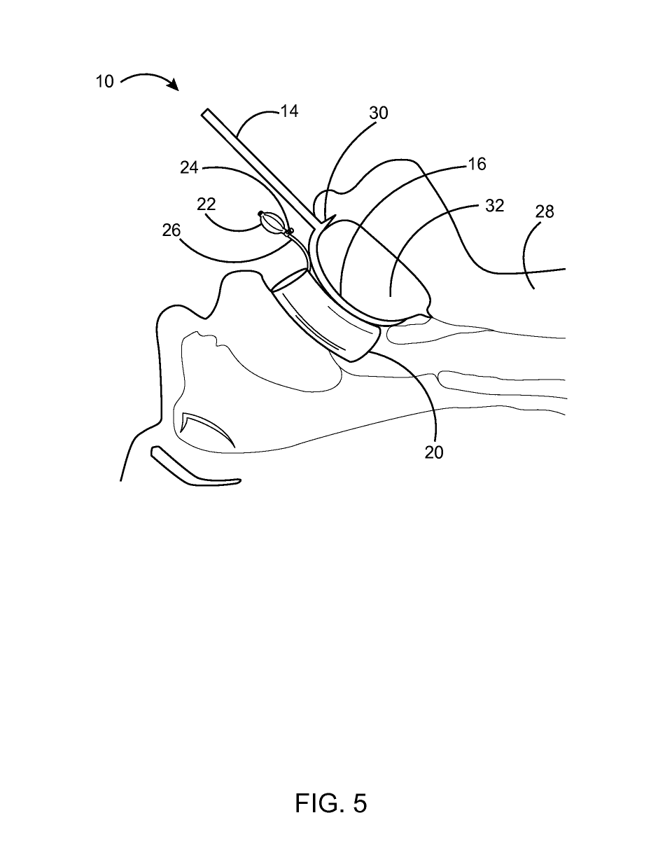

FIGS. 4-5 illustrate the airway opening device 10 in use wherein the device 10 is placed inside the mouth of an individual. Air is pumped into the inflation tube 20 to tighten the inflation tube 20 inside the mouth of an individual. Air is pumped into the inflation tube 20 through the tubing 26 and pressure screw control valve (not shown) by squeezing or compressing the inflation pump 22. The thumb screw 24 on the pressure screw control valve (not shown) also must be closed for the air pumped by the inflation pump 22 to inflate the inflation tube 20. If the thumb screw 24 is not closed, then air will exit the pressure screw control valve (not shown) through the thumb screw 24, rather than pass through the tubing 26 to inflate the inflation tube 20. Opening the thumb screw 24 will also release any air contained in the inflation tube 20 or the tubing 26. With the thumb screw 24 closed, air pumped by the inflation pump 22 through the pressure screw control valve (not shown) and the tubing 26 causes the inflation tube 20 to expand and tighten inside mouth of the patient 28. Air is continually pumped from the inflation pump 22 into the inflation tube 20 until a pressure level is reached. The curved portion 16 in the hard palate 12 covers and supports the tongue 32 of the supine patient 28. The perpendicular crest 30, provided at the junction of the curved portion 16 and the handle 14, will lodge behind the front teeth (in front of the tongue 32) for better stabilization of the device 10 when in operation. The inflating tubular component 18 pushes the curved portion 16 of the hard palate 12 against the tongue 32, thereby creating an airway and a passage for visualization of oropharynx and ideally the vocal cords.

When the usage of the airway opening device 10 is complete, the thumb screw 24 is completely opened to allow any air remaining within the inflation tube 20 to be released through the pressure screw control valve (not shown), thereby the device 10 gets deflated. Thereafter, the device 10 can be easily removed from the mouth.

Advantageously, the inflation passage has an internal cross-sectional area that is small enough such that it is comfortable for the patient 28 and facilitates minimal interference with the breathing of the patient 28 in use.

Advantageously, the inflatable tubular component 18 prevents obstruction of the airway of the patient 28 caused by the collapse of the airway in the nasopharyngeal region or in the upper oropharyngeal region, which may occur separately, or in addition to, collapse of the natural airway in the oropharyngeal region adjacent to the base of the tongue 32.

Advantageously, the present embodiment is effective in resisting the collapse of one or a combination of the nasopharyngeal wall, the oropharyngeal wall, the posterior pharyngeal wall, and the posterior region of the soft palate. As apnea and hypopnea during sleep, anesthesia or sedation can be caused by the collapse of one or a combination of the abovementioned tissues into the oropharynx, causing obstruction of the airway, the device 10 is therefore effective in maintaining the pharyngeal airway at least sufficiently open in use so that the patient 28 is able to continue breathing.

While a particular form of the invention has been illustrated and described, it will be apparent that various modifications can be made without departing from the spirit and scope of the invention. For example, the airway opening device 10 can be disposable and would be a preferred choice of production which will be not only more economical but also devoid of any risk of cross infection as may be the case in re-usable device. Accordingly, it is not intended that the invention be limited, except as by the appended claims.

What is claimed is:

1. An airway opening device for maintaining an airway in a supine patient, the airway opening device comprising:
a hard palate having a handle and a curved portion, wherein the curved portion covers and supports the tongue of the supine patient; and
an inflating tubular component attached to the curved portion of the hard palate, the inflating tubular component further comprising:
an inflation tube; and
an inflation pump attached to the inflation tube by means of a tubing, the inflation pump having a pressure screw control valve;
whereby the inflating tubular component pushes the curved portion of the hard palate against the tongue, thereby creating an airway.

2. The airway opening device of claim 1 wherein the curved portion lodges behind a front teeth for better stabilization of the airway opening device.

3. The airway opening device of claim 1 wherein the inflating tubular component pushes the hard palate against the tongue from below in the supine patient.

4. The airway opening device of claim 3 wherein the inflating tubular component pushes the hard palate from above thereby creating the inflation tube to maintain the airway.

5. The airway opening device of claim 1 wherein the inflation pump being preferably formed from a non-latex, polymeric material, such as polyvinyl chloride with a plasticizer for achieving a quick response from compressing and releasing the inflation pump.

6. An airway opening device for maintaining an airway in a supine patient, the airway opening device comprising:
a hard palate having a handle and a curved portion, wherein the curved portion covers and supports the tongue of the supine patient;
a perpendicular crest being situated at the junction of the curved portion, wherein the curved portion will lodge behind a front teeth for better stabilization of the airway opening device; and
an inflating tubular component attached to the curved portion of the hard palate, the inflating tubular component further comprising:
an inflation tube, wherein the inflation tube maintains the airway in a supine patient; and
an inflation pump attached to the inflation tube by means of a tubing, the inflation pump having a pressure screw control valve;
whereby the inflating tubular component pushes the curved portion of the hard palate against the tongue, thereby creating an airway.

7. The airway opening device of claim 6 wherein the perpendicular crest being lodged behind the front teeth for better stabilization of the airway opening device when in operation.

8. The airway opening device of claim 6 wherein the inflating tubular component pushes the hard palate against the tongue from below in the supine patient.

9. The airway opening device of claim 8 wherein the inflating tubular component pushes the hard palate from above thereby creating the inflation tube to maintain the airway.

10. An airway opening device for maintaining an airway in a supine patient, the airway opening device comprising:
a hard palate having a handle and a curved portion, wherein the curved portion covers and supports the tongue of the supine patient;
a perpendicular crest being situated at the junction of the curved portion, wherein the curved portion will lodge behind a front teeth for better stabilization of the airway opening device; and
an inflating tubular component attached to the curved portion of the hard palate, the inflating tubular component further comprising:
an inflation tube creates an inner hollow cavity in a supine patient; and
an inflation pump attached to the inflation tube by means of a tubing, the inflation pump having a pressure screw control valve;
whereby the inflating tubular component pushes the curved portion of the hard palate against the tongue, thereby creating an airway.

11. The airway opening device of claim 10 wherein the perpendicular crest being lodged behind the front teeth for better stabilization of the airway opening device when in operation.

12. The airway opening device of claim 10 wherein the pressure screw control valve prevents the air from escaping.

13. The airway opening device of claim 10 wherein the inflation pump being preferably formed from a non-latex, polymeric material, such as polyvinyl chloride with a plasticizer for achieving a quick response from compressing and releasing the inflation pump.

14. The airway opening device of claim 13 wherein the inflating tubular component pushes the hard palate against the tongue from below in the supine patient.

15. The airway opening device of claim 14 wherein the inflating tubular component pushes the hard palate from above thereby creating the inflation tube to maintain the airway.
